(19) United States
(12) Patent Application Publication
(10) Pub. No.: US 2010/0298861 A1
(43) Pub. Date: Nov. 25, 2010

(54) Delayed Action Spring Force Element for Nasal Dilators

(75) Inventor: Gustav R. Fenton, Minneapolis, MN (US)

Correspondence Address: GlaxoSmithKline;GLOBAL PATENTS -US, UW2220, P. O. BOX 1539, KING OF PRUSSIA, PA, 19406-0939, US

(73) Assignee: CNS, INC., Minneapolis, MN (US)

(21) Appl. No.: 12/092,954

(22) PCT Filed: Nov. 7, 2006
(86) PCT No.: PCT/US06/60587

§ 371 Date: Jul. 7, 2010

Related U.S. Application Data
(60) Provisional application No. 60/734,676, filed on Nov. 7, 2005.

(51) Int. Cl. A61M 29/00

(52) U.S. Cl. 606/199

(57) ABSTRACT

A nasal dilator, having a spring force that can be activated after the dilator is applied to the nose, is described. The nasal dilator has a spring force component and an adhesive component, which together act to dilate nasal tissues to help open or keep open the nasal valve to alleviate snoring and other breathing-related disorders.

This application claims the benefit of the filing date and contents of U.S. Provisional Patent application number 60/734,676, filed on Nov. 7, 2005.

### INTRODUCTION

External nasal dilators, useful for alleviating snoring and other breathing-related disorders, are called upon to perform two opposing functions. First, the dilator needs to attach securely to the skin of the nose. Second, the dilator applies outward force to open the nasal valve region. If the initial attachment force is insufficient then the second outward force will cause the dilator to detach. In current nasal dilators, adhesives, such as acrylate adhesives, set up for adherence relatively quickly such that the outward spring force does not typically cause detachment.

### BACKGROUND

There are, however, problems with the use of a pressure sensitive acrylate adhesive. For instance, the acrylate adhesive's strong adherence during removal (when less adherence would be kinder to the skin), its loss of adhesiveness during sweating, and its potential to block pores or cause irritation are all problems encountered when using fast-setting adhesives. While these issues are minor, they are widely acknowledged, since acrylate pressure sensitive adhesives are used extensively in commercially available wound care bandages.

For pressure sensitive adhesive applications, hydrocolloid adhesives are an alternative to acrylates. Initially, however, the hydrocolloids do not provide enough adhesiveness. As used herein, the term “adhesiveness” will be defined as the grams of force needed to remove the strip from the skin. The following is a comparison of typical adhesiveness over time:
Acrylate adhesiveness on human skin:
   ˜45 grams per inch initially,
   ˜100 grams per inch at four hours and
   ˜200 grams per inch at 24 hours.
Hydrocolloid adhesiveness on human skin:
   ˜25 grams per inch initially,
   ˜100 grams per inch at 20 to 30 minutes and
   ˜140 grams per inch at 24 hours.
      The acrylate adhesiveness (˜45 grams per inch) initially is adequate to counteract the external nasal dilator spring force, but the hydrocolloid's initial adhesiveness (˜25 grams per inch initially) is not sufficient to counteract the spring force. Between 5 and 20 minutes, the hydrocolloid's adhesiveness increases to a point where it can counteract the spring force.

Hydrocolloid materials offer good adhesion, ease of removal and gentleness to the skin. As noted above, the issue with a hydrocolloid adhesive is that its initial adhesiveness is insufficient to counteract the external nasal dilator's spring force, which can lead to detachment of the nasal dilator from the skin. The initial low adhesion builds to a significant amount as the hydrocolloid material warms up and adapts to the skin's surface, after which the adhesion is appropriate for countering the typical nasal dilator spring force. This period of growing adhesion may be anywhere from five minutes to an hour.

Therefore, there is a need for a nasal dilator adhesion system in which one could provide enough time for the hydrocolloid adhesive to establish adequate adhesion by delaying the outward detaching/dilating force that opens the nasal valve, i.e., the nasal dilator spring force.

### SUMMARY OF THE INVENTION

The present invention is directed to a nasal dilator for having a spring force element and an adhesive element. The spring force element has a latent state of minimal spring force, and can be activated to cause the spring force to increase to an amount sufficient to dilate nasal tissues.

### DESCRIPTION OF THE INVENTION

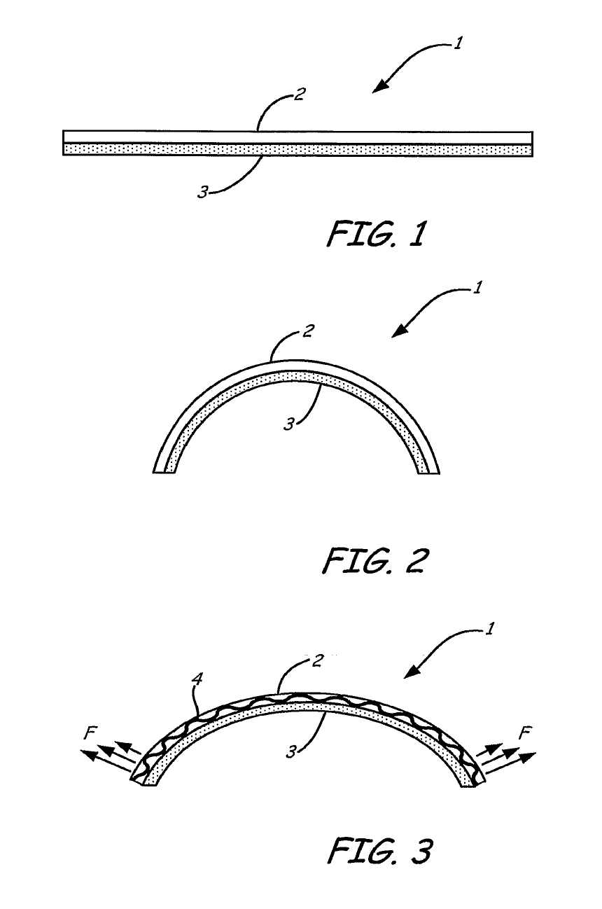

The present invention comprises two elements to form a nasal dilator strip 1, as shown in FIG. 1. The spring force layer 2 has minimal spring force in its latent, typically flattened state as shown in FIG. 1. The skin contact layer 3 provides a base for the adhesion to the nose and is most likely a hydrocolloid or similar material. An optional fabric or mesh framework 4, as shown in FIG. 3, can be incorporated into the spring force layer 2 to provide a lattice that can be both contracted and stiffened.

Although the embodiments contained herein describe these elements in the form of layers, other constructions, such as embedded elements, are within the scope of the present invention.

Nasal dilator strip 1 is attached to the nose by bending strip 1 to conform to the outer surface of the nose, as shown in FIG. 2. Upon some sort of activation process, the spring force layer 2 contracts, thereby gaining an outward spring force F sufficient to act as a nasal dilator, which is shown in FIG. 3. This activation would take place over a period preferably of about five to about thirty minutes after placing the nasal dilator strip 1 on the user's nose. This time period allows the skin contact layer 3 time to fully establish its adhesion to the nose skin prior to the outward force F reaching its maximum. Generally, adhesives that contract upon drying or curing can be used on the skin contact layer 3, in combination with materials in the spring force layer 2 that have the property of contracting upon exposure to air, light, moisture, heat, or a stress or pressure force, or any combination thereof.

The spring force layer 2 preferably has the following properties:
Intimately connected to the skin contact layer 3,
Minimal inherent lateral force when in its latent storage state and when first attached to the nose,
An activation mechanism or process whereby adequate lateral force (to open the nasal valve or otherwise dilate nasal tissues) can be developed, typically by contraction, within a period of about five to about thirty minutes after attachment to the nose,
Made of medical grade materials that are non-irritating to the skin,
Operates in an automatic manner such that the user does not need to intervene or intervenes minimally after starting the process by attaching the strip to his or her nose.

Several methods and embodiments are described below whereby the spring force layer 2 can be constructed to achieve the properties noted above.
Liner protection: The spring force layer 2 can be made of a liquid, gel or other material that is covered with a protective liner. To use the nasal dilator strip 1, the liner is removed, which exposes the spring force layer 2 to air or light. The exposure to air or light causes the material to dry or otherwise change and contract, which exerts outward force on the nose for dilation. An optional fabric or mesh framework 4, as shown in FIG. 3, can be incorporated into the spring force layer 2 to provide a lattice that can be both contracted and stiffened.
Encapsulation: The spring force layer 2 can be made of encapsulated liquid (for example, an adhesive) that could be activated by crushing the capsules and exposing the liquid. This embodiment is similar to “scratch and sniff” fragrance delivery systems. Once exposed, the encapsulated material contracts, causing the nose to dilate. An optional fabric or mesh framework 4, as shown in FIG. 3, can be incorporated into the spring force layer 2 to provide a lattice that can be both contracted and stiffened.
Bending stress activation: The spring force layer 2 can be made of material that is inactivate in its flattened storage state. The bending/stressing of the strip across the nose causes it to become activated to begin its contraction process. An optional fabric or mesh framework 4, as shown in FIG. 3, can be incorporated into the spring force layer 2 to provide a lattice that can be both contracted and stiffened.
Water activation: The spring force layer 2 can be made of material that is pliable under dry conditions, but contracts when it absorbs water. The water may be introduced by the user (for example, by wetting the outside of the strip after it is attached to the nose). Alternatively, a hydrocolloid material which absorbs water quite well and could act as a reservoir for the water to activate the spring force layer 2.
Heat activation: The spring force layer 2 can be made of material that is pliable under cool, storage conditions, but contracts when it is exposed to air and heated to body temperature. This embodiment may require a protective liner to be removed from the top of the spring force layer 2 to prevent activation during hot storage conditions. Alternatively, the strip can be sealed from access to air during storage.

From the previous description, it will be evident to those of skill in the art that the nasal dilator strip of the present invention can also be made of a single layer of material having both adhesive and contracting properties. The nasal dilator strip of the present invention can be made using any suitable materials and any conventional means that retain the desired properties of the strip when it is used by a consumer.

Although the foregoing fully describes and discloses the nasal dilator strip of the present invention, it is not intended to limit the scope of the present invention, which is defined by the following claims.

1. A nasal dilator for application to a nose to dilate nasal tissues, comprising a spring force layer and a skin contact layer.

2. The nasal dilator of claim 1, wherein the spring force layer includes a material having an initial latent state of minimal spring force and an active state of greater spring force.

3. The nasal dilator of claim 2, wherein the active state is achieved between about 5 to about 30 minutes after the nasal dilator is applied to the nose.

4. The nasal dilator of claim 2, wherein the greater spring force is sufficient to open a nasal valve and dilate nasal tissues.

5. The nasal dilator of claim 1, wherein the skin contact layer comprises an adhesive.

6. The nasal dilator of claim 5, wherein the adhesive has an initial low adhesiveness and a subsequent greater adhesiveness.

7. The nasal dilator of claim 6, wherein the initial low adhesiveness is sufficient to cause the nasal dilator to adhere to the nose.

8. The nasal dilator of claim 6, wherein the subsequent greater adhesiveness is sufficient for the nasal dilator to remain adhered to the nose upon dilation of the nasal tissues by the spring force layer.

9. The nasal dilator of claim 5, wherein the adhesive is a hydrocolloid adhesive.

10. The nasal dilator of claim 2, wherein the spring force layer further comprises a lattice capable of being contracted and stiffened.
